(19) United States
(12) Patent Application Publication
(10) Pub. No.: US 2018/0272112 A1
(43) Pub. Date: Sep. 27, 2018

(54) CONTINUOUS LOOP DRAINAGE SYSTEM DEVICE AND METHOD OF USE

(71) Applicant: Board of Regents, The University of Texas System, Austin, TX (US)

(72) Inventor: Ravi MURTHY, Houston, TX (US)

(21) Appl. No.: 15/761,498

(22) PCT Filed: Sep. 21, 2016
(86) PCT No.: PCT/US16/52917

§ 371 Date: Mar. 20, 2018

Related U.S. Application Data
(60) Provisional application No. 62/221,492, filed on Sep. 21, 2015.

(51) Int. Cl. A61M 27/00; A61M 25/00

(52) U.S. Cl. CPC A61M 27/002; A61M 25/0023; A61M 2202/0403; A61M 2206/20; A61M 2027/004; A61M 2205/8206; A61M 2210/106; A61M 2205/103; A61M 2205/3337

(57) ABSTRACT

Disclosed is a continuous loop drainage system. The continuous loop drainage system uses a double-lumen catheter to augment drainage of a body fluid, such as bile or urine, in a prograde direction through a closed-loop system. A miniature extracorporeal pump in the closed loop receives the fluid through an inflow lumen of the catheter and generates pressure in an outflow lumen of the catheter to cause flow of the fluid in a prograde direction distally through the outflow lumen toward the distal end of the catheter, such as the duodenum in the case of a biliary catheter or the urinary bladder in the case of a ureteral catheter. Other example uses are possible, such as in augmenting flow of cerebrospinal fluid through a ventriculoperitoneal shunt, for example.

### CROSS-REFERENCE TO RELATED APPLICATIONS

This application claims priority to U.S. Provisional Patent Application Ser. No. 62/221,492 filed Sep. 21, 2015, the contents of which are incorporated herein by reference.

### BACKGROUND OF THE INVENTION

### Technical Field

This invention relates to continuous loop drainage systems. In particular, the invention relates to a continuous loop biliary drainage catheter with an in-line pump to maintain prograde flow.

### State of the Art

Bile aids digestion in the small intestine and is produced continuously by the liver and delivered to the duodenum, which is the most proximal segment of the small intestine, by bile ducts. Bile is composed of water, bile salts, bilirubin, and fats. Excretion of bile into the small intestine is important for a functional liver and overall gastrointestinal health. Measurement of serum bilirubin and alkaline phosphatase, along with levels of other hepatocellular enzymes, are sensitive indicators of hepatic function, including the presence and degree of any biliary obstruction.

In some types of cancer, a locally expanding tumor mass may mechanically occlude a bile duct, blocking the drainage of bile into the duodenum. This tumor blockage may be intrinsic, via direct invasion of a bile duct wherein the tumor grows into the duct lumen; or extrinsic via compression by an adjacent expanding tumor mass. Regardless of the mechanism, if an obstructed bile duct prevents free-drainage of bile, liver cells (hepatocytes) upstream from the obstruction are damaged and impaired function of that portion of the liver eventually results.

If untreated, this blockage causes a variety of complications. One such complication is preclusion of chemotherapy administration to treat the underlying malignancy.

Biliary drainage catheters are an established and widely used means of providing biliary drainage when a bile duct becomes blocked. For approximately thirty percent (30%) of patients, currently available biliary drainage catheters are sufficient to relieve the obstruction and maintain biliary drainage from the obstructed liver tissue over at least several weeks to allow the patient to recover sufficient hepatic function to withstand chemotherapy. In seventy percent (70%), however, current drainage systems are inadequate to maintain drainage to the affected liver tissue for a sufficient time to allow functional recovery. Currently available biliary drainage catheters are problematic in that although bile will spontaneously flow retrograde through the catheter externally into a collecting reservoir, bile will not drain prograde into the duodenum, despite a patent and otherwise functional drainage catheter.

Accordingly, what is needed is a biliary drainage system that provides a structure and mechanism of maintaining patency for improved relief of biliary obstruction.

### DISCLOSURE OF EMBODIMENTS OF THE INVENTION

Certain embodiments include a continuous loop drainage system comprising: a catheter; and a pump, where: the catheter comprises a proximal end, a distal end, an inflow lumen, and an outflow lumen; the inflow lumen and the outflow lumen are fluidly coupled; and the pump is fluidly coupled to the proximal end of the catheter. Particular embodiments further comprise a pump control means configured to control operation of the pump. In specific embodiments, the inflow lumen has an inner diameter between 8 and 14 French. In some embodiments, the catheter is formed from a material with a negative surface charge and in certain embodiments the catheter is formed from a material with a positive surface charge.

In particular embodiments the inflow lumen comprises a plurality of holes. In some embodiments the catheter is approximately 50 centimeters in length. Specific embodiments further comprise a plurality of holes located along a length of approximately 30-35 centimeters of the inflow lumen. Certain embodiments comprise a bridge conduit fluidly coupled to the inflow lumen and to the outflow lumen. Particular embodiments further comprise a one-way valve in the bridge conduit, where: the one-way valve is configured to allow fluid flow from the inflow lumen to the outflow lumen; and the one-way valve is configured to prevent fluid flow from the outflow lumen to the inflow lumen.

In some embodiments the pump is a peristaltic pump configured to provide a flow capacity of between 0.5 milliliters per minute and 3.0 milliliters per minute. In specific embodiments the pump is configured to provide fluid flow in the outflow lumen from the proximal end of the catheter to the distal end of the catheter. In certain embodiments the pump powered by a battery, and in particular embodiments the battery is a lithium-hydride battery.

Certain embodiments include a method of facilitating prograde biliary drainage. In particular embodiments, the method comprises: placing a catheter in an obstructed bile duct, wherein the catheter comprises a proximal end and a distal end; traversing an obstruction in the obstructed bile duct with the catheter; positioning the catheter so that the distal end is located in a small intestine; and activating the pump coupled to the proximal end of the catheter, wherein bile passively drains retrograde to the proximal end of the catheter and is pumped prograde out of the distal end of the catheter.

In some embodiments of the method, the catheter comprises an inflow lumen and an outflow lumen, and the inflow lumen and the outflow lumen are fluidly coupled. In particular embodiments of the method, the pump is configured to provide fluid flow in the outflow lumen from the proximal end of the catheter to the distal end of the catheter. In specific embodiments of the method, the inflow lumen comprises a plurality of holes; the bile passively drains retrograde through the plurality of holes to the proximal end of the catheter; and the bile is pumped prograde through the outflow lumen out of the distal end of the catheter. In certain embodiments of the method, the catheter is approximately 50 centimeters in length and the plurality of holes are located along a length of approximately 30-35 centimeters of inflow lumen. In specific embodiments of the method, the catheter comprises a bridge conduit fluidly coupled to the inflow lumen and to the outflow lumen.

In certain embodiments of the method, the bridge conduit comprises a one-way valve, where: the one-way valve is configured to allow fluid flow from the inflow lumen to the outflow lumen; and the one-way valve is configured to prevent fluid flow from the outflow lumen to the inflow lumen. In particular embodiments of the method, the pump is a peristaltic pump configured to provide a flow capacity of between 0.5 milliliters per minute and 3.0 milliliters per minute. In some embodiments of the method, the catheter is positioned so that the distal end is located in a duodenum of the small intestine.

The foregoing and other features and advantages of the invention will be apparent to those of ordinary skill in the art from the following more particular description of the invention and the accompanying drawings.

Disclosed is a continuous loop drainage system device comprising a catheter, wherein the catheter comprises a proximal end, a distal end, an inflow lumen, and an outflow lumen wherein the inflow lumen and the outflow lumen are fluidly coupled; a pump, and a pump control means.

In some embodiments, the system device further comprises a negative pressure relief valve fluidly coupled to the inflow lumen.

Disclosed is a method of facilitating prograde biliary drainage comprising the steps of percutaneously placing a catheter in an obstructed bile duct; traversing an obstruction with the catheter; coupling the catheter to a pump; and activating the pump, wherein bile passively drains retrograde to a proximal end of the catheter wherein the proximal end is coupled to the pump and is pumped prograde out a distal end of the catheter wherein the distal end is located in a small intestine.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

FIG. 1 is a schematic representation of a continuous loop drainage system device inserted in a body;

FIG. 2 is a perspective view of a section of a double lumen catheter of a continuous loop drainage system device;

FIG. 3 is a perspective view of a pump 110 of a continuous loop drainage system device;

FIG. 4 is a perspective view of pump 110 of a continuous loop drainage system device;

FIG. 5 is a diagram of the steps of a method 200 of facilitating prograde biliary drainage.

FIG. 6 is a side view of an embodiment of a continuous loop drainage system device;

FIG. 7 is a contrast cholangiogram radiograph with an existing biliary drainage catheter; and

FIG. 8 is a contrast cholangiogram radiograph with the embodiment of FIG. 6.

### DETAILED DESCRIPTION OF EMBODIMENTS OF THE INVENTION

As discussed above, the disclosed invention relates to continuous loop drainage systems. In particular, the invention relates to a continuous loop biliary drainage system with an in-line pumping means to maintain prograde flow.

For patients with malignancies, such as primary or secondary liver cancer, primary bile duct cancer, pancreatic cancer, and the like, a percentage of affected individuals develop obstruction of a bile duct and consequently experience an accumulating serum bilirubin level. The increased level of bilirubin in the blood often leads to severe intractable, generalized itching (pruritus). An increasing serum bilirubin level is a contraindication to chemotherapy, therefore, a means of resolving the obstruction and draining the biliary tree is used, where possible, to allow serum bilirubin levels to decrease and stabilize. Stasis of bile in a biliary tree to which bacteria have been introduced through catheter placement and other drainage procedures is a risk factor for cholangitis and fatal biliary sepsis.

Medical providers, such as interventional radiologists, therefore, seek to relieve the obstruction by traversing the obstructed bile duct with a catheter, with or without mechanical dilation of the obstructed segment using a balloon or similar device, and then placing a device across the affected segment to maintain drainage of bile over a prolonged time period. Such biliary drainage systems include stents and biliary drainage catheters. Some biliary drainage catheters are placed percutaneously, wherein a medical specialist such as an interventional radiologist punctures the dilated bile duct upstream from the obstruction, usually through the liver itself, and inserts a drainage catheter prograde through the obstruction. The catheter end passing through the skin is connected to an external drainage bag and the opposite end within the bile duct or duodenum distal to the obstruction drains bile from the obstructed segment directly into the intestine.

For about thirty percent (30%) of patients, the standard biliary drainage catheter is sufficient to maintain patent biliary drainage for at least several weeks to allow liver function to recover and stabilize to a degree wherein administration of chemotherapy is possible. For the remaining seventy percent (70%) of patients, however the biliary drainage catheter does not remain functional for more than a very short period. Some catheters become internally obstructed with viscous bile or by rapid deposition of bile salt precipitates and other material causing a “crust” to form in the catheter lumen. More commonly, however, the viscosity of bile precludes passive drainage through the catheter into the duodenum, which is the most proximal segment of the small intestine and the site of the distal end of the drainage catheter. The absence of a pressure gradient between the intrahepatic bile ducts and the duodenum, combined with the relatively high viscosity and other characteristics of the bile as a fluid, effectively prevent the flow of bile from the obstructed biliary system out through the intestinal end of the catheter, regardless of the presence of a properly-positioned patent drainage catheter. Finally, in all patents with a percutaneously placed biliary drainage catheter, the patent must wear an external bag appliance to collect bile which does not spontaneously flow prograde into the intestine. An external bag appliance periodically must be emptied of bile, sometimes several times daily, is awkward and uncomfortable, and often substantially detracts from the quality of life for patients.

Typically, a minimum of maintaining biliary drainage for four-to-six weeks is necessary to allow recovery of hepatic function and administration of a course of chemotherapy. Current strategies for addressing early insufficient catheter prograde drainage include “upsizing” the drainage catheter with a larger device, however, these patients invariably return after only a few weeks with a blockage of the larger drainage catheter—insufficient time to allow for chemotherapy.

Ideally, a better strategy for maintaining prograde flow of biliary drainage through percutaneously placed catheters is needed.

Embodiments of the invention address this and other limitations by providing a continuous loop drainage system device which provides a means for continuous prograde drainage of bile from a percutaneously placed biliary catheter into the small intestine and eliminates the need for an external bag appliance. Embodiments of the device include systems which require no patient actions or monitoring for the device to function properly.

The elements of various embodiments of the invention include a dual-lumen catheter which is placed percutaneously and trans-hepatically into an obstructed intrahepatic bile duct. Like a standard percutaneously transhepatic cholangiocatheter (“PTC”), the catheter of the continuous loop drainage system device has a proximal end external to the skin and a distal end located in the patient's small intestine, typically in the duodenum, and comprises multiple holes in the catheter wall wherein bile from the bile duct surrounding the catheter proximal to the obstruction passes into a lumen of the catheter and may subsequently exit this lumen through additional holes located distal to the obstruction, whether in the more distal, unobstructed bile duct or the duodenum/small intestine. Also like a standard PTC, bile in the aforementioned lumen may flow retrograde into the portion of the catheter external to patient's body. Unlike any prior art catheter, however, bile refluxing in a retrograde direction remains in a continuous loop wherein an automatic pumping means augments prograde flow through a second catheter lumen out of a catheter distal end located in the duodenum/small intestine.

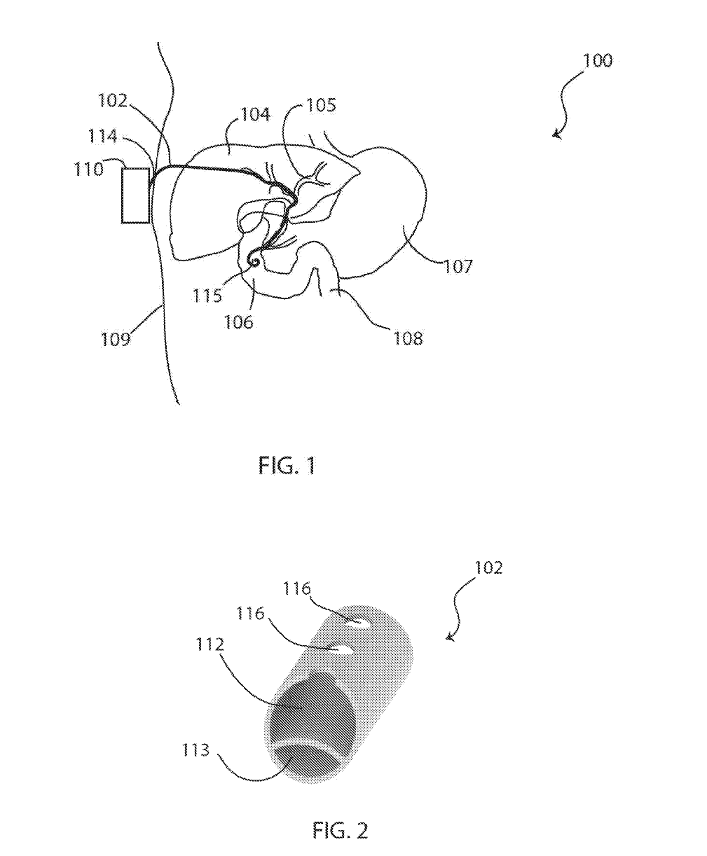

FIG. 1 is a schematic representation of a continuous loop drainage system device inserted in a body. FIG. 1 shows a closed loop drainage system 100 comprising a catheter 102 and a pump 110. Catheter 102 further comprises a proximal end 114 and a distal end 115. For the sake of clarity, FIG. 1 additionally shows some anatomical structures in relation to catheter 102, including a liver 104, a bile duct 105, a duodenum 106, a stomach 107, a small intestine 108, and a body wall 109.

Catheter 102, in some embodiments, is a double-lumen catheter, details of which are discussed herein below. Ideally, catheter 102 comprises physical characteristics that limit internal resistance to the flow of a fluid, particularly, fluid comprising bile. Bile is an emulsion of lipid micelles comprising lipid and cholesterol suspended in an aqueous base. The aqueous base, in turn, comprises a nearly saturated, saturated, or super-saturated solution of electrolytes and bile salts (salts of the weak bile acids). Bile is often a fluid with high viscosity.

Accordingly, minimizing internal resistance to flow within catheter 102 is accomplished, in some embodiments, by selecting a maximal internal diameter (“ID”) for inflow lumen 112 of catheter 102. In some embodiments, the ID of inflow lumen 112 measures 8 French. In some embodiments, the ID of inflow lumen 112 measures 10 French. In some embodiments, the ID of inflow lumen 112 measures 12 French. In some embodiments, the ID of inflow lumen 112 measures 14 French. In some embodiments, the ID of inflow lumen 112 measures 16 French. In some embodiments, the ID of inflow lumen 112 measures less than 8 French. In some embodiments, the ID of inflow lumen 112 measures greater than 14 French.

Minimizing internal resistance to flow within catheter 102 is accomplished, in some embodiments, by forming catheter 102 of a material with a low surface tension. In some embodiments, this is a material with a negative surface charge. In some embodiments, this is a material with a positive surface charge. In some embodiments, this is a material with a neutral surface charge. In particular embodiments, catheter 102 may be formed from polyurethane or silicone.

As shown by FIG. 1, proximal end 114 of catheter 102 is located external to body wall 109 and is coupled to pump 110. Conversely, distal end 115 is located in the gastrointestinal tract of a patient. In some embodiments, distal end 115 is located in a duodenum 106, which is the most proximal segment of the intestinal tract. In some embodiments, distal end 115 is located in a small intestine 106, with is anatomically the segment of the intestinal tract just distal to the duodenum. It is generally favorable to minimize the internal resistance of inflow lumen 112 to place distal end 115 in duodenum 106, wherein an overall length of inflow lumen 112 is minimized. The aforementioned locations of distal end 115 are by way of example only; in some embodiments, distal end 115 is located further distal in the intestinal tract of a patient's body. Catheter 102 is placed following percutaneous transhepatic cholangiography (“PTCA”) using standard techniques well known to those skilled in interventional radiology and related arts.

FIG. 2 is a perspective view of a section of a double lumen catheter of a continuous loop drainage system device. The segmental length of catheter shown in FIG. 2 comprises an inflow lumen 112, and outflow lumen 113. Inflow lumen 112 comprises a plurality of holes 116. In certain embodiments, catheter 102 may comprise an overall length of approximately 50 centimeters, and holes 116 may be located along a length of approximately 30-35 centimeters of inflow lumen 112. Following proper catheter placement shown in FIG. 1, holes 116 of inflow lumen 112 are present inside bile duct 105. Consequently, bile may flow from bile duct 105 into inflow lumen 112 by traversing holes 116. Bile may then flow in a prograde direction within inflow lumen 112 toward distal end 115, or in a retrograde direction within inflow lumen 112 toward proximal end 114. Whether bile flows in either direction, of remains static, depends upon the presence of a hydrostatic pressure gradient within inflow lumen 112 of catheter 102. If a pressure gradient exists and favors prograde flow, bile present within inflow lumen 112 flows towards distal end 115 and may exit inflow lumen 112 of catheter 102 into bile duct 105 distal to a tumor or other lesion obstructing bile duct 105 distal to the obstruction through a more distal hole 116 into a non-obstructed segment of bile duct 105 or, through the distal end of catheter 115 located in the duodenum, in some embodiments.

In some embodiments, catheter 102 comprises a third lumen (not shown in the figures), wherein third lumen receives a guidewire at distal end 115 of catheter 102 through the length of third lumen and exiting third lumen at a hole outside the body proximate to distal end 115 of catheter 102. Otherwise stated, a health care provider utilizes third lumen to pass catheter 102 into a body over a previously positioned guidewire using a well-established Seldinger technique known to those skilled in the art of catheter placement.

If the pressure gradient, however, favors retrograde bile flow, bile flows in a retrograde direction and may exit the body of the patient at proximal end 114. This is identical to a situation wherein a conventional PTC-placed single-lumen biliary drainage catheter is used, wherein the end of a conventional single-lumen catheter is coupled to an external drainage bag. Typically, a stopcock valve is present between the proximal end of a conventional catheter and an external drainage bag, wherein the valve may be manually opened to allow bile to drain into the external bag, which must be periodically emptied.

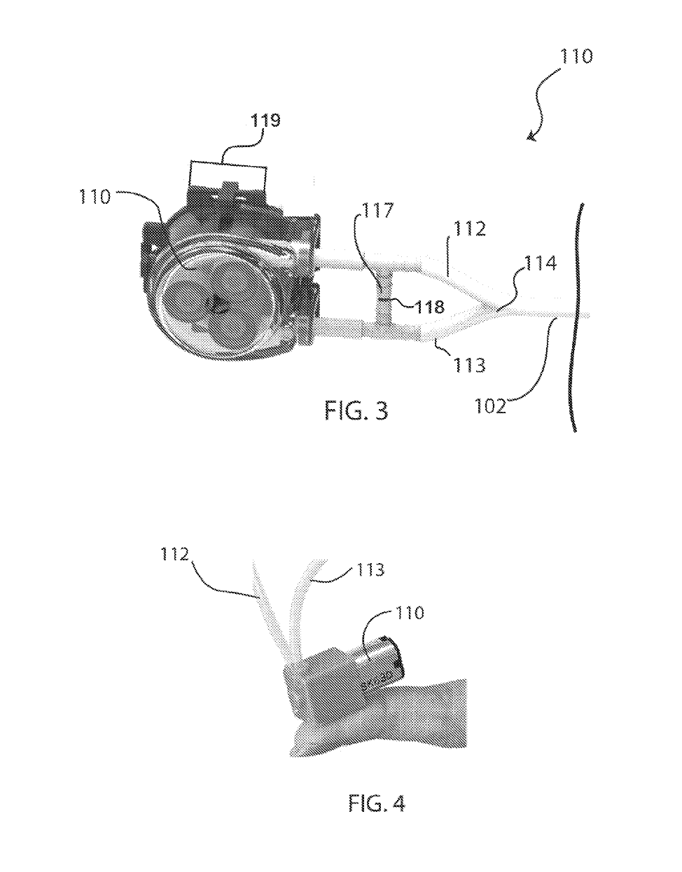

Continuous loop drainage system device 100, however, comprises proximal end 114 of catheter 102 coupled to a pump 110. FIG. 3 shows pump 110 coupled to inflow lumen 112 and also coupled to outflow lumen 113 at proximal end 114 of catheter 102. Pump 110 augments flow of bile from inflow lumen 112 through outflow lumen 113 and out distal end 115 under a condition wherein flow of bile within inflow lumen 112 is retrograde toward proximal end 114. Pump 110, in some embodiments, is a peristaltic self-priming roller pump, such as the example shown in FIG. 3. This is not meant to be limiting, however; other known and commercially available pump designs may be used. Pump 110 is powered by a battery 119, which may be a lithium-hydride battery or other suitable battery. Pump 110 comprises a minimum pump flow capacity of one (1) milliliter per minute (“ml/min.”). In some embodiments, pump 110 comprises a set pump rate. In some embodiments, pump 110 comprises a plurality of set pump rates. In some embodiments, pump 110 comprises a continuously variable pump rate of between 0.5 ml/min and 3 ml/min. In some embodiments, pump 110 comprises a pump rate of greater than 3 ml/min. In some embodiments, pump 110 operates discontinuously. In come embodiments, pump 110 operates continuously. In some embodiments, a flow rate of pump 110 is adjustable by a patient. In some embodiments, a patient may activate pump 110. In some embodiments, a patient may de-activate pump 110. In some embodiments, pump 110 is coupled to inflow lumen 112 and outflow lumen 113 of catheter 102 by a health care provider following placement of catheter 102 into a body. In some embodiments wherein catheter 102 comprises third lumen, pump 110 may be coupled to catheter 102 prior to insertion of catheter 102 into a body, whether by the health care provider who inserts catheter 102 into a body or during manufacture and packaging of catheter 102.

It is noted that pump 110 does not draw bile from inflow lumen 112 by creating a vacuum (negative pressure) within inflow lumen 112. Rather, under a condition where bile is available from inflow lumen 112, pump 110 operates by creating positive pressure inside outflow lumen 113 to cause bile to flow in a prograde direction within outflow lumen 113 toward and out from distal end 115 of catheter 102.

In some embodiments, a bridge conduit 117, as shown in FIG. 3, is coupled to inflow lumen 112 and outflow lumen 113 between proximal end 114 and pump 110. In embodiments where present, bridge conduit 117 functions to fluidly couple inflow lumen 112 and outflow lumen 113 under a condition wherein pump 110 is not activated; or, wherein pump 110 is activated however inflow from inflow lumen 112 is greater than the flow passing through pump 110 into outflow lumen 113. In some embodiments, bridge conduit 117 comprises a one-way valve 118 wherein flow of bile through bridge conduit 117 is in a direction from inflow lumen 112 to outflow lumen 113 and wherein the one-way valve prevents flow of bile from outflow lumen 113 to inflow lumen 112.

FIG. 4 is a perspective view of pump 110 of a continuous loop drainage system device. As shown in FIG. 4, pump 110, in some embodiments, comprises relatively small outside dimensions. Because of the small flow rates required to augment prograde flow of bile through a patent biliary drainage catheter into the duodenum, pump 110 may be a relatively diminutive device which is comfortably secured to the skin of a patient beneath a small dressing, or with a suture, and is not a bulky, high-profile device such as an external drainage bag. As mentioned herein above, pump 110 is a commercially available miniature peristaltic pump, in some embodiments. There are multiple advantages to embodiments wherein pump 110 is an extracorporeal miniature peristaltic pump. For example, it is not necessary to sterilize an extracorporeal pump. A peristaltic pump is self-priming and can effectively move a highly viscous or visco-elastic fluid without stalling, including fluid which may contain small pieces of solid material such as precipitated bile salts, necrotic tissue, blood clots, or cholesterol crystals, for example. A peristaltic pump is essentially noiseless.

FIG. 5 is a diagram of a method 200 of facilitating prograde biliary drainage. Method 200, as shown in FIG. 5, comprises an inserting step 210, and an activating step 220. Inserting step 210 comprising inserting a catheter, such as a double-lumen catheter with a plurality of holes in a wall of the catheter which communicate only with an inflow lumen, for example, into an obstructed intrahepatic bile duct; wherein a proximal end of the catheter remains outside the body and a distal end of the catheter is placed in a duodenum or small intestine. Inserting step 210 may be performed using well-established techniques, such as PTCA, known in the art. Activating step 220 comprises activating a pump, with or without a sensor, in some embodiments, coupled to proximal end of the catheter wherein the pump augments prograde flow of bile from the inflow lumen to an outflow lumen to the distal end of the catheter and into the duodenum or small intestine under a condition wherein bile flows retrograde through the inflow lumen of the catheter toward the proximal end of the catheter.

In some embodiments, method 200 further comprises a coupling step 230 wherein a pump is fluidly coupled between the inflow lumen and the outflow lumen of the catheter outside of a body at the proximal end of the catheter, completing a closed loop. In some embodiments, coupling step 230 is performed following insertion of catheter 102 into a body. In some embodiments, coupling step 230 is performed prior to insertion of catheter 102 into a body. In some embodiments, coupling step 230 is performed during manufacture of catheter 102.

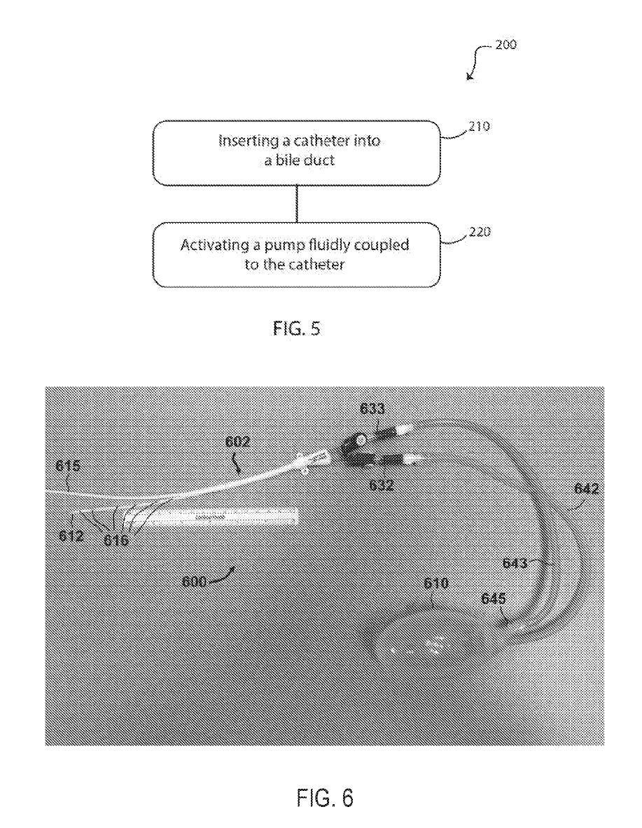

Referring now to FIG. 6, a continuous loop drainage system device 600 is shown with a dual lumen hemodialysis catheter 602 coupled to pumping mechanism 610. In this embodiment, pumping mechanism 610 is configured as a Jackson Pratt bulb with inflow (afferent) and outflow (efferent) unidirectional valved lumens (or limbs) 612 and 613, respectively. In this embodiment, inflow lumen 112 is configured as a hemodialysis silicone catheter arterial limb that comprises multiple oval holes 616 in the wall. During use, inflow lumen 612 will be placed in the liver, and holes 616 can allow bile to enter catheter 602 from different points along its length. Outflow lumen 613 does not comprise holes along its length, but includes a hole at distal end 615. During use, efferent limb 613 traverses the obstruction and terminates in the small intestine or bowel. Catheter ports 632 and 633 are in fluid communication with lumens 612 and 613, respectively, and are coupled to pumping mechanism 610 (e.g. with a luer-lock mechanism).

Pumping mechanism 610 comprises inflow (afferent) and outflow (efferent) unidirectional valved lumina 642 and 643, respectively. Lumina 642 and 643 are connected to the corresponding ports 632 and 633 on hemodialysis catheter 602. During use, a relief valve 645 can be opened and pumping mechanism 610 squeezed or compressed to expel room air. Relief valve 645 can then be closed while pumping mechanism 610 is still compressed, thereby creating a low continuous vacuum within the system. The valve on the outflow lumen is unidirectional, thereby preventing reflux of material in a retrograde fashion via the outflow or efferent portion of the system.

When catheter 602 is implanted and pumping mechanism 610 operated, bile within the obstructed system will decompress under low suction and fill the bulb in pumping mechanism 610 until the bulb regains its original shape (indicating that there is no residual suction in the system). The bulb is then squeezed manually to expel the contents via the outflow lumen 643 and 613 into the bowel and the process is repeated.

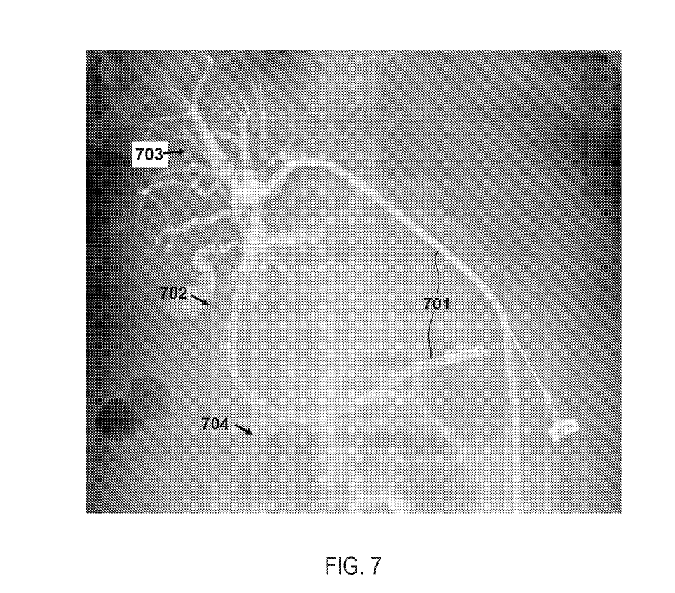

Referring now to FIG. 7, a contrast cholangiogram radiograph is shown with an existing biliary drainage catheter. A traditional silicone internal external biliary catheter 701 has been used to traverse a blockage 702 in the common bile duct of the liver in a patient with an indwelling metal endoprosthesis (metal stent).

The cholangiogram in FIG. 7 shows dilated contrast filled bile ducts 703 in the liver and absence of contrast in the small intestine 704. This is consistent with blockage of the traditional catheter with failure of the drainage system.

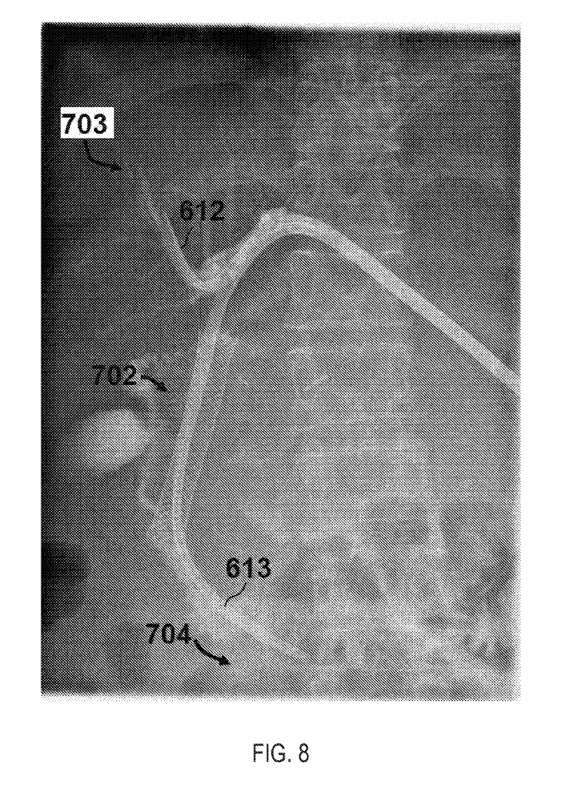

Referring now to FIG. 8 a contrast cholangiography radiograph is shown using an embodiment of catheter 602 from FIG. 6 in the same patient previously shown in FIG. 7. The inflow lumen (afferent limb) 612 of the system has allowed decompression of bile ducts 703 in the liver (note the reduced diameter of the ducts as compared to FIG. 7). The efferent limb 613 has traversed the obstruction 702 with contrast seen in small intestine 704.

This implies that bile has successfully been removed from the liver upstream to the obstruction 702 and replenished to its physiologic destination in the small intestine 704 using the mechanism of the proposed invention confirming proof of principle.

A continuous loop drainage system device and method of use is described. The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

1. A continuous loop drainage system comprising:
a catheter; and
a pump, wherein:
the catheter comprises a proximal end, a distal end, an inflow lumen, and an outflow lumen;
the inflow lumen and the outflow lumen are fluidly coupled; and
the pump is fluidly coupled to the proximal end of the catheter.

2. The continuous loop drainage system of claim 1 further comprising a pump control means configured to control operation of the pump.

3. The continuous loop drainage system of claim 1 wherein the inflow lumen has an inner diameter between 8 and 14 French.

4. The continuous loop drainage system of claim 1 wherein the catheter is formed from a material with a negative surface charge.

5. The continuous loop drainage system of claim 1 wherein the catheter is formed from a material with a positive surface charge.

6. The continuous loop drainage system of claim 1 wherein the inflow lumen comprises a plurality of holes.

7. The continuous loop drainage system of claim 1 wherein the catheter is approximately 50 centimeters in length.

8. The continuous loop drainage system of claim 7 further comprising a plurality of holes located along a length of approximately 30-35 centimeters of the inflow lumen.

9. The continuous loop drainage system of claim 1 further comprising a bridge conduit fluidly coupled to the inflow lumen and to the outflow lumen.

10. The continuous loop drainage system of claim 9 further comprising a one-way valve in the bridge conduit, wherein:
the one-way valve is configured to allow fluid flow from the inflow lumen to the outflow lumen; and
the one-way valve is configured to prevent fluid flow from the outflow lumen to the inflow lumen.

11. The continuous loop drainage system of claim 1 wherein the pump is a peristaltic pump configured to provide a flow capacity of between 0.5 milliliters per minute and 3.0 milliliters per minute.

12. The continuous loop drainage system of claim 1 wherein the pump is configured to provide fluid flow in the outflow lumen from the proximal end of the catheter to the distal end of the catheter.

13. The continuous loop drainage system of claim 1 wherein the pump powered by a battery.

14. The method of claim 13 wherein the battery is a lithium-hydride battery.

15. A method of facilitating prograde biliary drainage, the method comprising:
placing a catheter in an obstructed bile duct, wherein the catheter comprises a proximal end and a distal end;
traversing an obstruction in the obstructed bile duct with the catheter;
positioning the catheter so that the distal end is located in a small intestine; and
activating the pump coupled to the proximal end of the catheter, wherein bile passively drains retrograde to the proximal end of the catheter and is pumped prograde out of the distal end of the catheter.

16. The method of claim 15 wherein:
the catheter comprises an inflow lumen and an outflow lumen; and
the inflow lumen and the outflow lumen are fluidly coupled.

17. The method of claim 16 wherein the pump is configured to provide fluid flow in the outflow lumen from the proximal end of the catheter to the distal end of the catheter.

18. The method of claim 16 wherein:
the inflow lumen comprises a plurality of holes;
the bile passively drains retrograde through the plurality of holes to the proximal end of the catheter; and
the bile is pumped prograde through the outflow lumen out of the distal end of the catheter.

19. The method of claim 18 wherein the catheter is approximately 50 centimeters in length and the plurality of holes are located along a length of approximately 30-35 centimeters of inflow lumen.

20. The method of claim 16 wherein the catheter comprises a bridge conduit fluidly coupled to the inflow lumen and to the outflow lumen.

21. The method of claim 20 wherein the bridge conduit comprises a one-way valve, wherein:
the one-way valve is configured to allow fluid flow from the inflow lumen to the outflow lumen; and
the one-way valve is configured to prevent fluid flow from the outflow lumen to the inflow lumen.

22. The method of claim 15 wherein the pump is a peristaltic pump configured to provide a flow capacity of between 0.5 milliliters per minute and 3.0 milliliters per minute.

23. The method of claim 15 wherein the catheter is positioned so that the distal end is located in a duodenum of the small intestine.
